(19) United States
(12) Patent Application Publication
(10) Pub. No.: US 2024/0041524 A1
(43) Pub. Date: Feb. 8, 2024

(54) SYSTEMS FOR VALVE RESECTION AND RESHAPING CATHETER/LASER-BASED VALVE LEAFLET REMOVAL

(71) Applicant: KONINKLIJKE PHILIPS N.V., EINDHOVEN (NL)

(72) Inventors: Nathan C FRANCIS, COLORADO SPRINGS, CO (US); Michael ANDERSON, COLORADO SPRINGS, CO (US); Kenneth Peter GRACE, WOODLAND PARK, CO (US)

(21) Appl. No.: 18/266,383

(22) PCT Filed: Dec. 16, 2021
(86) PCT No.: PCT/EP2021/086048

§ 371 Date: Jun. 9, 2023

Related U.S. Application Data
(60) Provisional application No. 63/129,458, filed on Dec. 22, 2020.

(51) Int. Cl. A61B 18/24

(52) U.S. Cl. CPC A61B 18/24; A61B 2018/00369

(57) ABSTRACT

A leaflet resection catheter apparatus and system for a Transcatheter Aortic Valve Replacement (TAVR) procedure including a catheter configured at a distal end with a guidewire to deploy the catheter within a vessel lumen to a situs of an aortic valve for leaflet resection; an accessory tool configured with a set of grasping elements attached at a catheter's distal end to enable the accessory tool to travel down the vessel lumen to the situs of the aortic valve to exert a pulling action to draw the aortic valve leaflet in a direction towards the catheter's distal end to draw a portion of the aortic valve leaflet into a protective sleeve of the catheter at the catheter's distal end; and one or more fibers of a ring of fibers surrounding the catheter's distal end to resect tissue that includes a portion of the aortic valve leaflet drawn into the protective sleeve.

### TECHNICAL FIELD

The technical field generally relates to endovascular procedures and more particularly relates to systems and methods for use in a Transvenous/transcatheter Aortic Valve Replacement (TAVR) procedure.

### BACKGROUND

Transcatheter aortic valve replacement (TAVR) is a minimally invasive heart procedure to replace a narrowed aortic valve that fails to open properly (i.e., aortic valve stenosis). The transcatheter aortic valve replacement is also called transcatheter aortic valve implantation (TAVI). The treatment by transvenous and transcatheter aortic valve repair (TAVR) is becoming more prevalent and accepted as a treatment option for patients because widespread training has made available the treatment option to medical personnel. Hence, more medical personnel have gained first-hand knowledge of this newer advanced medical procedure and recognize the patient benefits that can be realized.

The TAVR procedure allows for the implanting (i.e., replacing) of a heart valve without having to open the chest cavity. The resultant minimal invasive surgery for a heart valve replacement makes surgical valve replacement are more feasible treatment plan. This is because TAVR can now be considered an option for patients considered at intermediate or high risk of complications from traditional open-chest surgical aortic valve replacement.

However, there are differences in both approaches; for example, in the open chest replacement surgery, the degraded valve is completely removed. In TAVR, the damaged, native valve is left in place. The valve can have anatomical abnormalities, calcification, or infection. However, inserting a new valve over the native valve can cause complications in the TAVR procedure, including valve migration, valve embolization, paravalvular leakage, and blockage of the coronary arteries restricting blood flow to the heart. These complications can also occur, sometimes more frequently, during a valve-in-valve TAVR, where the transcatheter valve is deployed within a previously implanted bioprosthetic valve.

Therefore, deploying a TAVR valve on top of the existing damaged valve, either native or bioprosthetic, may not be performed because of the expected complications in the procedure. To alleviate the complications from implantation of a new valve, a catheter may be used to remove old valve leaflets at the location to prepare the implant site for a cleaner valve deployment and operation.

It is desired for a catheter configured to remove the old valve leaflets and prepare the site for the new valve replacement procedure.

Accordingly, technologically improved systems and methods for valve resection and reshaping using a catheter with an ablation tool to perform valve leaflet removal in a TAVR procedure is desirable. The following disclosure provides these technological enhancements, in addition to addressing related issues.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

In one exemplary embodiment, an apparatus including a leaflet resection catheter for a Transcatheter Aortic Valve Replacement (TAVR) procedure is provided. The apparatus includes a catheter configured at a distal end with a guidewire to deploy the catheter within a vessel lumen to a situs of an aortic valve for leaflet resection; an accessory tool configured with a set of grasping elements attached at a catheter's distal end to enable the accessory tool to travel down the vessel lumen to the situs of the aortic valve to exert a pulling action to draw the aortic valve leaflet in a direction towards the catheter's distal end to draw a portion of the aortic valve leaflet into a protective sleeve of the catheter at the catheter's distal end, and one or more fibers of a ring of fibers surrounding the catheter's distal end to resect tissue that includes a portion of the aortic valve leaflet drawn into the protective sleeve at the catheter's distal end.

Furthermore, other desirable features and characteristics of the system and method will become apparent from the subsequent detailed description and the appended claims, taken in conjunction with the accompanying drawings and the preceding background.

### BRIEF DESCRIPTION OF THE DRAWINGS

The present application will hereinafter be described in conjunction with the following drawing figures, wherein like numerals denote like elements, and:

FIG. 1 is an illustration of an exemplary diagram of combination tool of an apparatus for use in a TAVR procedure of the guidewire lumen, accessory tool, and pull-push action laser cutting catheter implemented by the TAVR system, in accordance with an exemplary embodiment;

FIG. 2 illustrates an exemplary diagram of another embodiment of a separate apparatus for use in a TAVR procedure of a combination of an accessory tool and cutting catheter implemented by the TAVR system, in accordance with an embodiment; and

FIG. 3 illustrates an exemplary flowchart for a method system of a leaflet resection catheter for use in a TAVR procedure, in accordance with an exemplary embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments. The embodiments described herein are exemplary embodiments provided to enable persons skilled in the art to make or use the invention and not to limit the scope of the invention that is defined by the claims. Furthermore, there is no intention to be bound by any expressed or implied theory presented in the preceding technical field, background, summary, or the following detailed description.

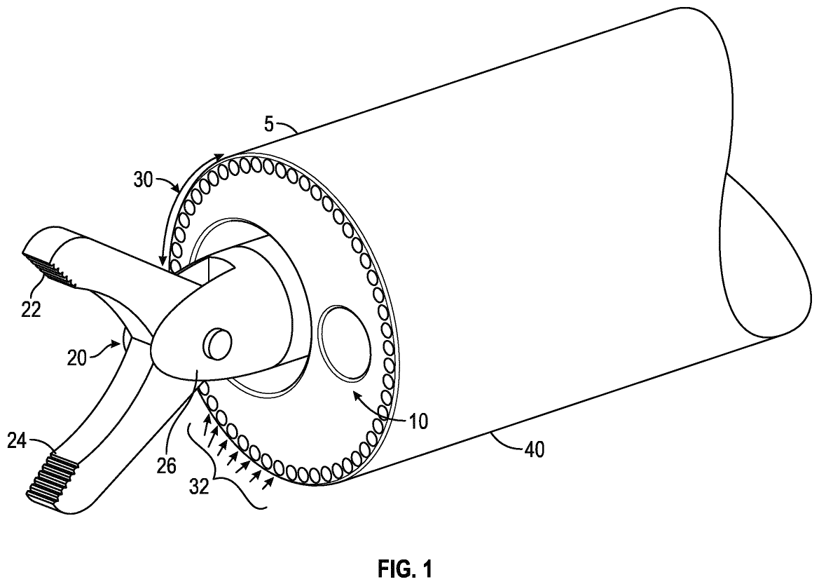

Exemplary embodiments provide a technical solution to this problem in the form of a leaflet resection catheter deployment system for use in a TAVR procedure (FIG. 1) embodying novel rules, vascular anatomy design factors, and recommended treatment protocols for deploying a combination apparatus including a catheter, accessory tool, and catheter cutting apparatus in a vessel lumen, as follows:

Provided embodiments include an improved catheter-based procedure for deploying a new TAVR valve on top of an existing damaged valve by the medical personnel gaining access to the heart valve, and inserting the catheter, and tracking it to the position. The catheter cutting apparatus is configured to excise valve leaflets and prepare the site for valve replacement. The outer sheath of the catheter is pulled back, exposing the grasping catheter to grab the leaflet tissue, and to draw the ring of fibers to ablate the leaflet from either the native or bioprosthetic valve.

Provided embodiments disclosed enable the performance of a medical procedure using an accessory tool for the forwardly grasping of tissue (i.e., the leaflets). The accessory tool is configured as a grasping catheter that can enable the user to pull a grasped leaflet into a catheter sheath and the excising of the leaflet tissue within the cutting sheath to prevent damage to surrounding tissue.

The figures and descriptions below provide more detail.

Turning now to FIG. 1, in an embodiment, the system for a leaflet resection catheter deployment by a catheter system 100 for use in a TAVR procedure in a vessel (also referred to herein as “system” 100) is depicted as associated with a vessel lumen (not shown). In various embodiments, the vessel lumen is a blood vessel in a patient. As mentioned, system 100 embodies bidirectional motion to perform tissue ablation of tissue with a catheter sheath at a situs in a vessel lumen. The catheter 5 may be manually operated by a user; manual input can include a direction and a placement operation.

The direction is generally, from the perspective of the distal tip of the catheter 5, forward and aft, longitudinally, within a vessel lumen. To perform TAVR, the doctor may access your heart through a blood vessel in your leg or through a tiny incision in your chest. The doctor may use other approaches to access your heart. A hollow tube (catheter 40) is inserted through the access point. Your medical provider can use various advanced imaging techniques to guide the catheter 40 through a vessel lumen to the heart valve location for rescission of the leaflet tissue at the valve location. The deployment system 100 includes a guidewire lumen 10, an accessory tool 20, and a opposite direction laser cutting catheter 30. FIG. 1 illustrates an exemplary embodiment of the distal tip of the catheter system 100, featuring a guidewire lumen 10 to track the catheter 40 to the valve (not shown) in question, accessory tool lumen for the grasping catheter to travel to the valve in question, and in this embodiment, a ring of fibers for laser transmission to ablate the leaflet tissue from either the native or bioprosthetic valve.

The accessory tool 20 includes proximal elements 22, 24 (or grasping elements) and a distal element 26, which protrudes outward from the catheter 40. The proximal elements 22, 24 are coupled together on the distal side to enable the proximal elements to be positionable on opposite sides of the leaflets (not shown) to capture or retain the leaflets therebetween.

The mitral valve is composed of two leaflets, the anterior leaflet, which is a semi-circular shape and attaches to two-fifths of the annular circumference. There is continuity between the anterior leaflet of the mitral valve and the left and non-coronary cusp of the neighboring aortic valve, referred to as the aortic-mitral curtain. These two components of the aorto-mitral curtain are on two separate anatomical planes, situated at an angle of 120°, which corresponds to the planes of the aortic and mitral annulus, respectively, which can be grasped because of the angular location by the proximal elements 22, 24. When the flaps (i.e., leaflets) of the mitral valve do not close tightly enough, it causes blood to leak backward into the left atrium. This occurs due to valve leaflets bulging back—a condition called mitral valve prolapse and can be corrected by the valve replacement. The proximal elements 22, 24 may be made of cobalt-chromium, nitinol, or stainless steel, and the distal elements 26 can also be made of cobalt-chromium and stainless steel, or another material. In the alternative exemplary embodiment, the accessory tool can be a vacuum suction (instead of proximal elements) to grasp the aortic valve leaflet during the ablation of tissue of the aortic valve leaflet. The accessory tool is configured to pass through an introducer (not shown) of at least a range of 14f to 18f for use with a standard TAVR deployment catheter. In an exemplary embodiment, a sheath introducer is a long, wide bore, single lumen catheter with a wide plastic hub on the proximal end, which has a central smaller hole (one-way-valved to prevent back-flow of blood), through which various other vascular catheters can be inserted. In an exemplary embodiment, the sheath introducer comes in multiple diameters and lengths.

The laser cutting catheter 30 includes a ring of fibers 32 in a plurality of arrangements of an entirety, a semicircle, a one-third circle, or another fractional circle of the circumference of the catheter 40 distal end to ablate tissue of the aortic valve leaflet. The laser cutting catheter 30, in another exemplary embodiment, can reshape a valvular structure to better fit a replacement valve at the situs of the valve replacement. Also, a power source (not shown) is connected at a proximal end of the catheter 40 to energize a laser (via the fibers 32) to ablate tissue of the aortic valve leaflet grasped by the proximal elements 22, 24.

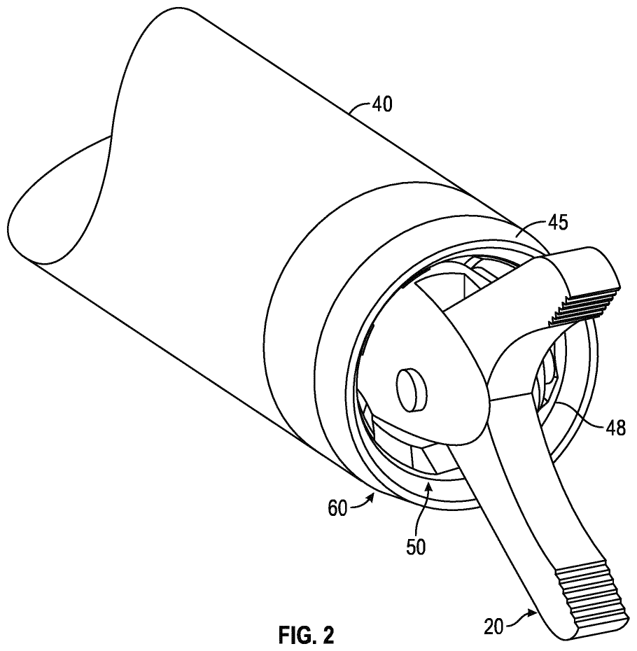

Turning to FIG. 2, an exemplary diagram of another embodiment of the stent deployment system is illustrated in accordance with an embodiment. In FIG. 2, The main components for the invention are a cutting sheath 50 to resect the valve leaflets, any type of power source (not shown) to energize the cutting mechanism on the cutting sheath 50, and a grasping catheter (or accessory tool) 20 to travel down the lumen (interior of cutting sheath 50) of the cutting sheath and grasp the leaflets. In FIG. 2, the cutting sheath 50 catheter 40 could also be a manual or powered mechanical cutter within a protective sleeve 45 to prevent unwanted tissue damage during deployment. The outer cutting sheath 48 will have a hemostatic valve (i.e., a valve to keep blood within the vessel lumen or to stop any bleeding) on the proximal end to allow tool pass-through while sealing off arterial pressures if an arterial approach is used. The catheter 40 will need to pass through a 14F-18F introducer, which is standard for a TAVR deployment catheter 40. In the exemplary embodiment, a vacuum suction instead of the grasping accessory tool 20 could be used to grasp, or retain the valve leaflet during cutting by the cutting sheath 50. In an exemplary embodiment, the cutting sheath 50 can also be implemented to reshape a valvular structure to better fit a replacement valve at the location in the vessel lumen. The catheter 40 can also be inserted via a transvenous approach or transapical approach.

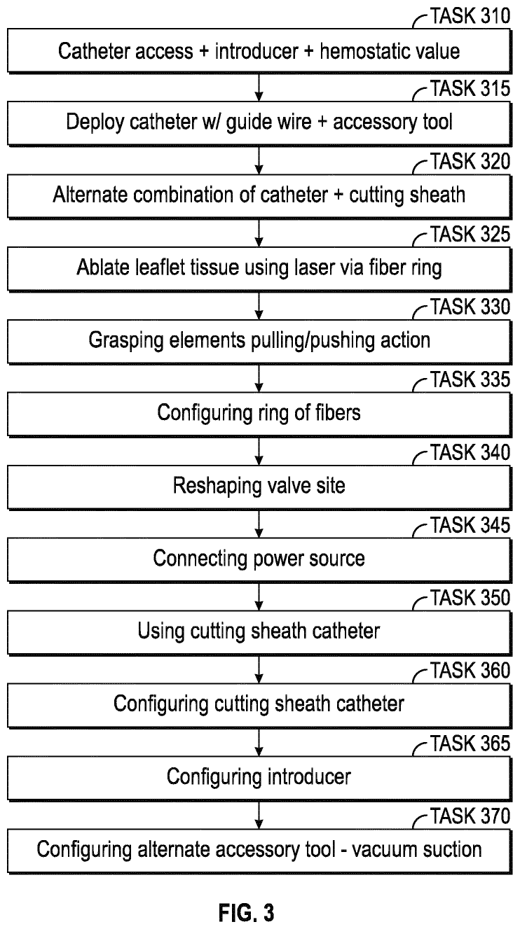

FIG. 3 illustrates an exemplary flowchart of the TAVR method for using the catheter configured with the accessory tool and cutting sheath in accordance with various embodiments. In an exemplary embodiment, FIG. 3 illustrates a method for deployment of a combination catheter, accessory tool, and cutting sheath structure or laser ablation tool in a vessel lumen. At task 310, the user (i.e., healthcare provider) gains access to an artery or vein via an introducer. Also, the catheter can be configured with a hemostatic valve on the proximal end to enable the accessory tool to pass through the vessel lumen whilst sealing off arterial pressures. At task 315, the user inserts the catheter that is configured at a distal end with a guidewire for deploying the catheter within a vessel lumen to a situs of an aortic valve for leaflet resection. The catheter at task 315 is also configured with an accessory tool of a set of grasping elements attached at the catheter's distal end to enable the accessory tool to travel down the vessel lumen to the situs of the aortic valve. The accessory tool enables the grasping of an aortic valve leaflet between the set of grasping elements of the accessory tool at the catheter's distal end to exert a pulling action to draw the aortic valve leaflet in a direction towards the catheter's distal end.

In an alternative exemplary embodiment, at task 320, the user inserts a catheter configured at a distal end with an accessory tool including a set of grasping elements attached at a catheter's distal end to enable the accessory tool to travel down a vessel lumen to a situs of the aortic valve. The accessory tool grasps an aortic valve leaflet between the set of grasping elements of the accessory tool at the catheter's distal end to exert a pulling action to draw the aortic valve leaflet in a direction towards the catheter's distal end. Further, the accessory tool by the pulling action draws a portion of the aortic valve leaflet into a protective sleeve of the catheter at the catheter's distal end. The cutting sheath catheter resects tissue of a portion of the aortic valve leaflet drawn into the protective sleeve at the catheter's distal end. The tissue resection is of tissue of a portion of the aortic valve leaflet that has been drawn within the protective sleeve and held between the set of grasping elements.

At task 325, one or more fibers transmit the laser to ablate tissue whilst the aortic valve leaflet is held by the set of grasping elements within the protective sleeve, thereby preventing tissue damage outside the protective sleeve. At task 330, in response pulling action of the accessory tool while grasping an aortic valve leaflet tissue between the set of grasping elements, enabling a reactive action of simultaneous pulling the leaflet in a direction into the protective sleeve whilst moving the distal end of the catheter in the opposite direction towards the aortic valve leaflet that is drawn into the protective sleeve at the catheter's distal end. At task 335, the ring of fibers about a circumference of the catheter's distal end in entirety or part of enable the ablating of tissue of the aortic valve leaflet. At task 340, laser transmitted via the fibers enables the reshaping of a valvular structure to better fit a replacement valve at the situs. At task 345, a power source is connected at a proximal end of the catheter to energize the laser for ablating the tissue of the aortic valve leaflet.

In an alternative exemplary embodiment, at task 350, the cutting sheath catheter is powered mechanical cutter located within the protective sleeve to prevent unwanted tissue damage.

At task 360, the cutting sheath catheter is configured as an outer cutting sheath with a hemostatic valve on a proximal end of the catheter to enable the accessory tool to pass through the vessel lumen whilst sealing off arterial pressures. At task 365, the accessory tool is configured to pass through an introducer of at least a range of 14f to 18f for use with a standard TAVR deployment catheter.

At task 370, in an alternate exemplary embodiment, the catheter is configured with an accessory tool that includes a vacuum suction to grasp the aortic valve leaflet during the ablation of tissue of the aortic valve leaflet.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps.

However, it should be appreciated that such block components (or modules) may be realized by any number of hardware, software, and/or firmware components configured to perform the specified functions. To clearly illustrate the interchangeability of hardware, various illustrative components, blocks, modules, and steps have been described above generally in terms of their functionality. Whether such functionality is implemented as hardware or software depends upon the application and design constraints imposed on the overall system.

Skilled artisans may implement the described functionality in varying ways for each application, but such implementation decisions should not be interpreted as causing a departure from the scope of the present invention. In addition, those skilled in the art will appreciate that the embodiments described herein are merely exemplary implementations.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. When “or” is used herein, it is the logical or mathematical or, also called the “inclusive or.” Accordingly, A or B is true for the three cases: A is true, B is true, and A and B are true. In some cases, the exclusive “or” is constructed with “and;” for example, “one from the set A and B” is true for the two cases: A is true, and B is true.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

What is claimed is:

1. An apparatus comprising a leaflet resection catheter for a Transcatheter Aortic Valve Replacement (TAVR) procedure, the apparatus comprising:
a catheter configured at a distal end with a guidewire to deploy the catheter within a vessel lumen to a situs of an aortic valve for leaflet resection;
an accessory tool configured with a set of grasping elements attached at a catheter's distal end to enable the accessory tool to travel down the vessel lumen to the situs of the aortic valve to exert a pulling action to draw an aortic valve leaflet in a direction towards the catheter's distal end to draw a portion of the aortic valve leaflet into a protective sleeve of the catheter at the catheter's distal end; and
one or more fibers of a ring of fibers surrounding the catheter's distal end to resect tissue that comprises a portion of the aortic valve leaflet drawn into the protective sleeve at the catheter's distal end.

2. The apparatus of claim 1, further comprising:
the one or more fibers for the ring of fibers configured to transmit a laser to ablate tissue whilst the aortic valve leaflet is held by the set of grasping elements within the protective sleeve to prevent tissue damage outside the protective sleeve.

3. The apparatus of claim 1, further comprising,
the accessory tool configured to, in response pulling action while grasping an aortic valve leaflet tissue between the set of grasping elements, enable a reactive action of a simultaneous pulling of the leaflet in a direction into the protective sleeve whilst the distal end of the catheter is motioned in an opposite direction towards the aortic valve leaflet that is drawn into the protective sleeve at the catheter's distal end.

4. The apparatus of claim 3, further comprising:
the accessory tool is configured to comprise a vacuum suction for grasping the aortic valve leaflet during ablation of tissue of the aortic valve leaflet.

5. The apparatus of claim 3, further comprising:
the ring of fibers configured about a section or an entirety of the catheter's distal end to ablate tissue of the aortic valve leaflet.

6. The apparatus of claim 4, further comprising:
the ring of fibers configured in a plurality of arrangements comprising at least a semicircle, a one-third circle, or another fractional circle of a circumference of the catheter's distal end to ablate tissue of the aortic valve leaflet.

7. The apparatus of claim 3, further comprising:
the ring of fibers configured to cause the situs, which comprises a valvular structure to reshape to better fit a replacement valve at the situs.

8. The apparatus of claim 3, further comprising:
a power source connected at a proximal end of the catheter configured to energize a laser for ablating the tissue of the aortic valve leaflet.

9. The apparatus of claim 1, further comprising:
the set of grasping elements of the accessory tool at the catheter's distal end is configured to grasp an aortic valve leaflet between each grasping element of the set of grasping elements.

10. The apparatus of claim 1, wherein the aortic valve leaflet comprises a native and a bioprosthetic valve leaflet.

11. The apparatus of claim 1, further comprising:
a hemostatic valve configured on a proximal end of the catheter to enable the accessory tool to pass through the vessel lumen whilst sealing off arterial pressures.

12. The apparatus of claim 1, further comprising:
the accessory tool is configured to pass through an introducer of at least a range of 14f to 18f for use with a standard TAVR deployment catheter.

13. The apparatus of claim 1, further comprising:
each fiber of a fiber ring transmits laser energy configured to perform tissue resection of tissue, which comprises a portion of the aortic valve leaflet that has been drawn within the protective sleeve and held between the set of grasping elements.

14. A system using a leaflet resection catheter in a Transcatheter Aortic Valve Replacement (TAVR) procedure in a vessel lumen, comprising:
a catheter configured with an accessory tool at a distal end to enable resecting leaflet tissue at a valve situs for preparing a situs for valve replacement;
the catheter configured at the distal end with a guidewire for deploying the catheter within the vessel lumen to the situs of the valve for leaflet resection;
wherein the accessory tool comprises a set of grasping elements attached at a catheter's distal end to enable the accessory tool to grasp a leaflet between the set of grasping elements at the catheter's distal end and to exert a pulling action to draw the leaflet in a direction towards the catheter's distal end;
in response to the pulling action of the leaflet, a portion of the leaflet drawn into a protective sleeve of the catheter at the catheter's distal end; and
wherein a ring of fibers surrounding the catheter's distal end configured to resect tissue of the portion of the leaflet drawn into the protective sleeve at the catheter's distal end by one or more fibers wherein each fiber of a fiber ring transmits laser energy to perform tissue resection of the leaflet portion contained within the protective sleeve.

15. The system of claim 14, wherein the one or more fibers is configured to ablate via transmissions of the laser to tissue whilst the leaflet is held by the set of grasping elements within the protective sleeve, thereby preventing tissue damage outside the protective sleeve.

16. The system of claim 15, further comprising,
in response to the pulling action of the accessory tool, while grasping leaflet tissue between the set of grasping elements, the catheter configured to exhibit a pull-push action to simultaneous pull the leaflet in a direction towards the protective sleeve whilst moving the distal end of the catheter in an opposite direction towards the leaflet and drawing the protective sleeve over the leaflet at the catheter's distal end.

17. The system of claim 16, further comprising:
the ring of fibers configured about a circumference of a catheter's distal in entirety to ablate tissue of the leaflet.

18. The system of claim 17, further comprising:
the ring of fibers configured about a part of the catheter's distal to ablate tissue of the leaflet.

19. The system of claim 18, further comprising:
the ring of fibers configured in a plurality of arrangements that comprise at least a semicircle, a one-third circle, or another fractional circle of the circumference of the catheter's distal end to ablate tissue of the leaflet.

20. An apparatus using a leaflet resection catheter in a Transcatheter Aortic Valve Replacement (TAVR) procedure in a vessel lumen, comprising:
a catheter configured with an accessory tool at a distal end to enable resecting leaflet tissue at a valve situs for preparing a situs for valve replacement;
the catheter configured at the distal end with a guidewire for deploying the catheter within the vessel lumen to the situs of the valve for leaflet resection;
wherein the accessory tool comprises a set of grasping elements attached at a catheter's distal end to enable the accessory tool to grasp a leaflet between the set of grasping elements at the catheter's distal end and to exert a pulling action to draw the leaflet in a direction towards the catheter's distal end;
in response to the pulling action of the leaflet, a portion of the leaflet drawn into a protective sleeve of the catheter at the catheter's distal end; and
wherein a ring of fibers surrounding the catheter's distal end configured to resect tissue of the portion of the leaflet drawn into the protective sleeve at the catheter's distal end by one or more fibers wherein each fiber of a fiber ring transmits laser energy to perform tissue resection of the leaflet portion contained within the protective sleeve.
